(19) United States
(12) Patent Application Publication
(10) Pub. No.: US 2003/0114381 A1
(43) Pub. Date: Jun. 19, 2003

(54) Method of treating sensorineural hearing loss

(76) Inventors: Douglas A. Cotanche, Southborough, MA (US); Mark A. Parker, Hull, MA (US)

Correspondence Address: NIXON PEABODY LLP, 101 FEDERAL ST, BOSTON, MA, 02110, US

(21) Appl. No.: 10/274,180

(22) Filed: Oct. 18, 2002

Related U.S. Application Data
(60) Provisional application No. 60/346,238, filed on Oct. 22, 2001.

(51) Int. Cl. A61K 38/00

(52) U.S. Cl. 514/12

(57) ABSTRACT

The present invention provides a method for treating and preventing sensorineural hearing loss in a subject in need of such treatment by implanting stem cells into the subject's inner ear. The stem cells of the present invention are preferably derived from a neuronal lineage and are capable of differentiating into cochlear hair cells. In the preferred embodiment of the invention, the subject suffering from the sensorineural hearing loss is a mammal such as a human.

### CROSS-REFERENCE TO RELATED APPLICATIONS

The present application claims benefit of U.S. Provisional Application No. 60/346,238, filed on Oct. 22, 2001.

### FIELD OF THE INVENTION

The present invention generally relates to the treatment of hearing loss disorders, and more particularly to the treatment of sensorineural hearing loss.

### BACKGROUND OF THE INVENTION

In the United States, almost thirty million people exhibit some level of deafness or hearing loss. While this disability is rarely life threatening, it impairs more people psychologically and economically than epilepsy, multiple sclerosis, spinal injury, stroke, and Huntington's and Parkinson's diseases combined (Hudspeth, 1997). The vast majority of the cases of hearing impairment are caused by a loss of hair cells, the sensory cells in the inner ear that transduce sounds into neural signals. This type of hearing loss is called “sensorineural hearing loss” and is caused by a number of factors including sound trauma, ototoxic drug exposure, disease, viral infection, and genetic disorders. Severe to profound sensorineural hearing loss affects one in every thousand children born in the United States country, with half of these due to hereditary causes (Rehm & Morton, 1999).

In most cases of severe to profound hearing loss the cochlear nerve is still intact. For these individuals hearing function can often be restored with an electronic cochlear implant. After successful cochlear implantation, most patients can recognize environmental sound, understand spoken language, listen to music and in many cases use the telephone. However, there are significant limitations to this technology. Occasionally individual electrodes fail within the ear, requiring programming with a more limited number of electrodes. There is a small percentage of total device failures, which require complete surgical replacement of the device. The inner ear electrodes are placed very near the facial nerve, and in some patients the desired level of electrical stimulation cannot be used because it stimulates the nerve and causes disturbing facial twitching. Moreover, for some implant models the internal device must be removed completely or in part if the patient ever needs MRI scanning. Additionally, in a small minority of patients the device simply fails to provide much sound information to the brain. While even patients with a “poor” result usually acquire valuable information about environmental sound, a few patients struggle to understand speech or other more complex sounds.

Most importantly, even in patients with the most ideal result, cochlear implantation never provides hearing that approaches the frequency specificity and sophistication of normal hearing. There are many reasons for this, but the foremost is that the hair cells of the inner ear do not simply “hear” sound; they also act as small motors that actively “tune” the inner ear to hear sound with greater sensitivity and frequency specificity. Unlike the cochlear implant that electrically stimulates a region of the cochlea and thereby stimulates many auditory nerve fibers at once, hair cells have the capability to stimulate one auditory nerve fiber at a time. This allows much more detailed information about sound to be transmitted to the brain.

Due to the myriad of disadvantages associated with an expensive, low-fidelity, battery-powered, programmed external devices that are currently available for the hearing-impaired, methods that actually treat sensorineural hearing loss and thus enable people to regain their hearing without the use of electronic devices are needed. Hair cell regeneration has been hypothesized as a potential way to bypass the need for such devices in humans ever since it was first discovered over a decade ago in the avian auditory system (Cotanche, 1999; Stone & Rubel, 2000).

It has been shown that the normal postnatal mammalian cochlea does not regenerate hair cells, and that there is a time window during embryonic development when additional hair cells can be induced to develop in the immature cochlea. Studies by Matthew Kelley and colleagues (Kelley et al., 1993, 1995) have shown that the undifferentiated progenitor cells that will give rise to all of the cell types within the organ of Corti can be induced by retinoic acid to form supernumerary hair cells between embryonic days 13 and 16. Furthermore, when existing hair cells are killed by laser ablation during this same developmental period, adjacent uncommitted progenitor cells change their fates and differentiate into hair cells, replacing those that were lost. Surprisingly, labeling experiments with markers for DNA synthesis indicate that in each of these cases the additional and/or replacement hair cells do not arise from proliferation of the existing cells. Rather, it appears that existing progenitor cells within the developing organ of Corti are able to change their developmental fates in response to changes in their local environment. Thus, at least for a short time during mammalian development, the cochlea contains progenitor cells that have the ability to develop into many different cell types.

To date, the isolation and propagation of cochlear progenitor cells has proved elusive. However, cell lines derived from stem cells present a more practical source of cells for transplantation. The therapeutic potential presented by the use of stem cells is profound. Consequently, several murine and human stem cell lines have been generated as tools of scientific inquiry. One such cell line, derived from the inner cell mass of blastocyst stage embryos, is capable of generating cloned embryonic stem cells (ES) (Martin, 1981; Evans and Kaufman, 1981). Cell lines derived from these cells have a seemingly unlimited proliferation potential but are also capable of differentiating after re-introduction into an intact organ. Reimplantation studies suggest that ESCs are totipotent, retaining the ability to develop as any cell type within the body (Martin, 1981; Evans and Kaufman, 1981). More recently, transplantation of ESCs into adult organ systems has demonstrated that these cells will respond to existing environmental signals in a manner similar to progenitor cells, leading to the development of organ specific cell types (Brüstle et al., 1999; McDonald et al., 1999; Learish et al., 1999). Therefore, ESCs present a potential source of cells for transplantation into the organ of Corti.

The number of studies examining the potential use of ESCs for regenerative transplantation is rapidly increasing. Notably, the murine C17.2 neural stem cell (NSC) line, which is derived from the fetal cerebellum, has been used in several transplant experiments involving the brain and spinal cord. The C17.2 clonal cell line exhibits constitutive expression of the lacZ gene product E. Coli β-galactosidase (β-gal{tilde over ())} which enables the transplanted NSCs to be identified histochemically using 5-bromo-4-chloro-3-indolyl D-L-galactosidase (X-gal) (Snyder et al., 1995). Several studies have indicated that transplanted NSCs can integrate into the site of a brain lesion and differentiate into native cell types. Transplantation of these NSCs replaced deficient oligodendrocytes in the shiverer mouse, a strain that lacks MBP-expressing oligodendrocytes (Yandava et al., 1999).

Snyder et al., showed that NSCs have the ability to integrate into a site of lesion and replace a degenerative cell type; that NSCs may be used to replace cells that normally do not exhibit regeneration; and that NSCs have migratory capacity (Snyder et al., 1997). In the study, specific cell types in the adult murine cortex were subjected to targeted apoptosis by photolytic degeneration followed by C17.2 cell line transplantation into the brain. The NSCs migrated to the site of lesion and differentiated into pyramidal cortical neurons and glia, effectively replacing the targeted cells and reversing neuronal loss. Upon transplantation, C17.2 derived NSCs exhibit a profound proficiency to migrate throughout the brain and body. In a study that inserted intracranial tumors into one hemisphere of the mouse brain, C17.2 derived NSCs transplanted in the ipsilateral hemisphere migrated to and were incorporated within the tumor body (Aboody et al., 2000). Furthermore, NSCs transplanted into the contralateral hemisphere migrated across the corpus callosum to infiltrate the tumor. Astonishingly, NSCs transplanted into the tail vein of these mice traveled to the brain, crossed the blood-brain barrier, and infiltrated the tumor mass. In addition, human correlates of C17.2 cells injected into the ventricular system of non human primates resulted in NSC migration from the ventricles as far as layer I of the sensorimotor cortex (Ourednik et al 2001). These studies highlight not only the ability of NSCs to differentiate into various cell types, but the ability of the NSCs to migrate through many tissues as well.

### SUMMARY OF THE INVENTION

The present invention provides a method for treating and preventing sensorineural hearing loss in a subject in need of such treatment by implanting stem cells into the subject's inner ear. The stem cells of the present invention are preferably derived from a neuronal lineage and are capable of differentiating into cochlear hair cells. In the preferred embodiment of the invention, the subject suffering from the sensorineural hearing loss is a mammal such as a human.

The method of the present invention further provides for a transplantation site that is flexible and need not be the site of lesion.

The terms “to transplant,” “to implant,” “to deliver,” and “to administer” are used interchangeably herein throughout and mean the act of introducing neural stem cells into the subject's inner ear.

### DESCRIPTION OF THE INVENTION

We have surprisingly discovered that stem cells can be used to regenerate cell types that normally do not regenerate, such as cochlear hair cells. We have discovered that administration of stem cells, particularly neural stem cells (NSCs), in a mouse model, causes regeneration of cochlear hair cells and restores hearing. The NSCs used in the method of the present invention differentiate into cochlear hair cells, integrate into the sensory epithelia, form neural linkages with the existing cochlear nerves and thereby provide sound information to the brain and hearing to the subject. Thus, the present invention presents a method for treating a subject having or prone to a sensorineural hearing impairment or treating a mammal prophylactically to prevent or reduce the occurrence or severity of a sensorineural hearing impairment that would result from inner ear cell injury, loss, or degeneration, such as that caused by a cytotoxic agent, by administering a therapeutically effective amount of stem cells that promote hair cell regeneration, growth, proliferation, or prevent or reduce cytotoxicity of hair cells.

The subjects targeted for treatment by the current invention are mammals, and preferably humans. The subjects include those subjects with inner ear hair cell related conditions, and preferably sensorineural hearing loss. Tests are known and available for diagnosing hearing impairments. Neuro-otological, neuro-ophthalmological, neurological examinations, and electro-oculography can be used. (Wennmo et al Acta Otolaryngol (1982) 94:507-15). Sensitive and specific measures are available to identify patients with auditory impairments. For example, tuning fork tests can be used to differentiate a conductive from a sensorineural hearing loss and determine whether the loss is unilateral. An audiometer is used to quantitate hearing loss, measured in decibels. With this device the hearing for each ear is measured, typically from 125 to 8000 Hz, and plotted as an audiogram. Speech audiometry can also be performed. The speech recognition threshold, the intensity at which speech is recognized as a meaningful symbol, can be determined at various speech frequencies. Speech or phoneme discrimination can also be determined and used an indicator of sensorineural hearing loss since analysis of speech sounds relies upon the inner ear and 8th nerve. Tympanometry can be used to diagnose conductive hearing loss and aid in the diagnosis of those patients with sensorineural hearing loss. Electrocochleography, measuring the cochlear microphonic response and action potential of the 8th nerve, and evoked response audiometry, measuring evoked response from the brainstem and auditory cortex, to acoustic stimuli can be used in patients, particularly infants and children or patients with sensorineural hearing loss of obscure etiology. These tests serve a diagnostic function as well as a clinical function in assessing response to therapy.

Sensory and neural hearing losses can be distinguished based on tests for recruitment (an abnormal increase in the perception of loudness or the ability to hear loud sounds normally despite a hearing loss), sensitivity to small increments in intensity, and pathologic adaptation, including stapedial reflex decay. Recruitment is generally absent in neural hearing loss. In sensory hearing loss the sensation of loudness in the affected ear increases more with each increment in intensity than it does in the normal ear. Sensitivity to small increments in intensity can be demonstrated by presenting a continuous tone of 20 db above the hearing threshold and increasing the intensity by 1 db briefly and intermittently. The percentage of small increments detected yields the “short increment sensitivity index” value. High values, 80 to 100%, is characteristic of sensory hearing loss, whereas a neural lesion patient and those with normal hearing cannot detect such small changes in intensity. Pathologic adaptation is demonstrated when a patient cannot continue to perceive a constant tone above the threshold of hearing; also known as tone decay. A Bekesy automatic audiometer or equivalent can be used to determine these clinical and diagnostic signs; audiogram patterns of the Type II pattern, Type III pattern and Type IV pattern are indicative of preferred hearing losses suitable for the treatment methods of the invention. As hearing loss can often be accompanied by vestibular impairment, vestibular function can be tested, particular when presented with a sensorineural hearing loss of unknown etiology. When possible, diagnostics for hearing loss, such as audiometric tests, should be performed prior to exposure in order to obtain a patient normal hearing baseline. Upon exposure, particularly to an ototoxic drug, audiometric tests should be performed twice a week and continued testing should be done even after cessation of the drug treatment since hearing loss may not occur until several days after cessation. U.S. Pat. No. 5,546,956, provides methods for testing hearing that can be used to diagnose the patient and monitor treatment. U.S. Pat. No. 4,637,402, provides a method, for quantitatively measuring a hearing defect, that can be used to diagnose the patient and monitor treatment.

The stem cells useful according to the present invention include cells that are capable of differentiating into cochlear hair cells in a subject suffering from sensorineural deafness so as to replace defective cochlear hair cells and restore hearing. The stem cells include, but are not limited to, neural or embryonic stem cells, fetal pig or other xenotropic stem cells, and bone marrow derived stem cells. Preferred stem cells for use in the present invention include NSCs, embryonic stem cells that have been modified to express cochlear progenitor cell markers, or cell lines established from umbilical chord or adult neural tissue. Snyder et al., U.S. Pat. No. 5,598,767, discloses methods for isolating NSCs. Preferably, 0.5 to 2 μl of stem cells in a phosphate buffer solution is delivered into the tissues surrounding the cochlear duct. The “buffer” may be any suitable buffer that is generally regarded as safe and generally confers a pH from or about 4.8 to 8, preferably from or about 5 to 7. Examples include acetic acid salt buffer, which is any salt of acetic acid, including sodium acetate and potassium acetate, succinate buffer, phosphate buffer, citrate buffer, histidine buffer, or any others known to the art to have the desired effect.

According to the method of the invention, stem cells are implanted into the subject's inner ear. The site of the stem cell implantation, according to the present invention, is flexible and, therefore, can be a site other than the site of lesion. In the method of the present invention, stem cells can be implanted by any method known in the art, for example, into the scala media via basal turn penetration, into the scala tympani via round window penetration, or into the spiral ganglion via round window penetration.

Generally, delivery of therapeutic agents in a controlled and effective manner with respect to tissue structures of the inner ear (e.g., those portions of the ear contained within the temporal bone which is the most dense bone tissue in the entire human body) is known. Exemplary inner ear tissue structures of primary importance include but are not limited to the cochlea, the endolymphatic sac/duct, the vestibular labyrinth, and all of the compartments which include these components. Access to the foregoing inner ear tissue regions is typically achieved through a variety of structures, including but not limited to the round window membrane, the oval window/stapes footplate, and the annular ligament. The middle ear can be defined as the physiological air-containing tissue zone behind the tympanic membrane (e.g. the ear drum) and ahead of the inner ear. It should also be noted that access to the inner ear may be accomplished through the endolymphatic sac/endolymphatic duct and the otic capsule. The inner ear tissues are of minimal size, and generally accessible through microsurgical procedures.

Delivery of therapeutic cells to the inner ear of a subject can be done by contact with the inner ear or through the external auditory canal and middle ear, as by injection or via catheters, or as exemplified in U.S. Pat. No. 5,476,446, which provides a multi-functional apparatus specifically designed for use in treating and/or diagnosing the inner ear of a human subject. The apparatus, which is useful in the practice of the present invention, has numerous functional capabilities including but not limited to (1) delivering therapeutic agents into the inner ear or to middle-inner ear interface tissues; (2) withdrawing fluid materials from the inner ear; (3) causing temperature, pressure and volumetric changes in the fluids/fluid chambers of the inner ear; and (4) enabling inner ear structures to be electro-physiologically monitored. In addition, other systems may be used to deliver the factors and formulations of the present invention including but not limited to an osmotic pump which is described in Kingma, G. G., et al., “Chronic drug infusion into the scala tympani of the guinea pig cochlea”, Journal of Neuroscience Methods, 45:127-134 (1992). An exemplary, commercially-available osmotic pump may be obtained from the Alza Corp. of Palo Alto, Calif. (USA). U.S. Pat. No. 4,892,538, provides an implantation device for delivery of the factors and formulations of the invention. NSCs or other stem cells that have been modified to express cochlear progenitor cell can be implanted in the subject to provide the therapeutic effect.

The effectiveness of treating hearing impairments with the methods of the invention can be evaluated by the following signs of recovery, including recovery of normal hearing function, which can be assessed by known diagnostic techniques including those discussed herein, and normalization of nerve conduction velocity, which is assessed electro-physiologically.

The references cited throughout the specification are herein incorporated in their entirety. The present invention is further illustrated by the following examples and claims. The examples are provided to aid in the understanding of the invention and are not to be construed as a limitation thereof.

### EXAMPLES

Identification of Specific Cellular Phenotypes

Cochlear hair cells are identified using phalloidin and/or a number of recently identified markers that have been shown to be specific for hair cells. These include antibodies against myosin VI and myosin VIIa (Avraham et al., 1995; Hasson et al., 1997).

Confocal Microscopy

Cochlear cultures or whole mount samples from intact cochleae are fixed by immersion for 1 hour in 4% paraformaldehyde in phosphate buffered saline (PBS) at pH 7.4. The tissues are permeablized in 1% Triton X-100 in PBS, rinsed, and blocked in 10% normal goat serum (NGS). Samples are incubated in one, two or three primary antibodies for 2 hours at RT. For triple label immunocytochemistry the cochlear tissues are incubated in appropriate secondary antibodies tagged with either fluorescein, rhodamine, or Texas Red for 2 hours at RT. If less than three primary antibodies are used, the third channel can be used to label F-actin by incubating for 30 minutes in Texas Red-conjugated phalloidin (1:200; Molecular Probes, Eugene, Oreg.) in PBS. Samples are mounted in Vectashield (Vector Laboratories, Burlingame, Calif.) onto glass microscope slides. Confocal scanning laser microscopy is performed on a Leica TCS SP confocal laser scanning microscope equipped with epifluorescence optics (Leica Microsystems Heidelberg GmbH, Heidelberg, Germany). This microscope has three lasers for simultaneous viewing of three different fluorescent labels plus a fourth channel for DIC imaging of the sensory epithelium. Optical Z-series scans through the entire thickness of the cochlear epithelia are made using either a 20× (NA 0.7), 40× (NA 1.25) or 63× (NA 1.2) water immersion objective or a 100× (NA 1.4) oil immersion lens.

Functional Surgical Procedures

Animals will be anesthetized with ketamine (100 mg/kg i.p.) and xylazine (20 mg/kg I.p.), with booster injections (⅓ original dose) as needed. ABRs will be recorded without any surgical intervention. Surgical preparation for round window recordings and DPOAEs involves tracheostomy and removal of the right pinna, after which distortion-product otoacoustic emissions (DPOAEs) will be measured. Then, the right bulla will be exposed and opened with a #11 scalpel for round window recordings.

Testing of ABRs

ABR potentials will be evoked with tone pips and recorded via needle electrodes inserted through the skin (vertex to ipsilateral pinna near tragus with a ground on the back near the tail). Stimuli will be 5-ms pips (0.5-ms rise-fall with a cos² onset envelope, delivered at 40/sec). The response will be amplified (10,000 ×), filtered (100 Hz-3 kHz), and averaged with an A-D board in a LabVIEW-driven data-acquisition system. Sound level will be raised in 5 dB steps from roughly 10 dB below threshold up to 80 dB SPL. At each sound level, 1024 responses will be averaged (with stimulus polarity alternated). The software includes an “artifact reject” feature in which response waveforms will be discarded if the peak-to-peak voltage exceeds 15 μV. ABR “thresholds”, defined as the lowest sound level at which the response peaks are clearly present, will be read by eye from stacked waveforms obtained at 5 dB sound pressure intervals (up to 80 dB SPL). Thresholds typically correspond to a level one step below that at which the peak-to-peak response amplitude begins to rise.

Testing of DPOAEs

The DPOAEs will be measured using an ER-10C (Etymotics Research) acoustic system consisting of two sound sources and one microphone. The sensitivity of the microphone (dB volts/dB SPL) is measured by coupling a calibrated Bruel and Kjaer condenser microphone to the output port of the ER-10C system. Stimuli consist of two primary tones (f₁:f₂=1:2), presented with f₂ level always 10 dB<f₁ level. Stimuli are generated digitally, but attenuation will be provided with analog attenuators. The ear canal sound pressure is filtered (high pass at 1000 Hz), amplified, and digitized by a D-A board. A FFT will be computed, and the sound pressures at f₁, f₂ and 2f₁−f₂ are extracted after spectral averaging from serial waveform traces. The noise floor also is measured (defined as the average of 6 points in the FFT on either side of the 2f₁−f₂ frequency) and ranged between −20 and 5 dB SPL, depending on frequency.

Round Window Recordings

Electro-mechanical activity is recorded from the ears of each mouse via a silver wire on the round window referred to the tongue. The response is amplified (10,000×), filtered (100 Hz-3 kHz), and averaged with an A-D board in a LabVIEW-driven data acquisition system. Response thresholds are measured under computer control in response to 5-ms tone pips (0.5-ms rise-fall with a cos² onset envelope, delivered at 10/sec). At each SPL, 32 responses is averaged (with stimulus polarity flipped on half of the presentations to remove microphonic potentials). Threshold is defined as the sound pressure required that produces a peak-to-peak neural response of 3 μV.

Stem Cell Cultures

C17.2 NSC cultures are generated and maintained as described in Snyder et al., 1992. Briefly, C17.2 cells are grown at 37° C. in Dulbecco's Modified Eagle Medium supplemented with 10% fetal bovine serum, 10 mM sodium pyruvate, 2 mM glutamine, and 100 U each of penicillin, streptomycin, and amphotericin B per ml. Stable undifferentiated C17.2 clones encoding the lacZ vector are trypsinized from a 90% confluent dish and resuspended in phosphate-buffered saline at 4×10⁴ cells/μl. 0.5 to 2.0 μl drop of this suspension is drawn into a 10 μl Hamilton syringe fixed to a glass micropipette to be used for transplantation into recipient mice.

Vertebrate Animals

Mice are utilized as the recipients for the stem cell transplants. Particularly, we used the 3-week-old female mice of the CD-1 strain of mice obtained from Charles River Labs. Veterinary care of the animals is provided by the BID ARF (Animal Research Facility). The mice are anesthetized during stem cell implant procedures and during auditory function testing. They are anesthetized with either 2-4% isofluorane by inhalation or with ketamine (80-100 mg/kg)/xylazine (10 mg/ml) by IP injection, with booster injections (⅓ original dose) as needed. Mice will be euthanized with a 0.1 to 0.3 ml IP injection of Beuthanasia (pentobarbital/phenytoin). This is consistent with the recommendations of the 2000 Report on the AVMA Panel on Euthanasia.

Transplantation Studies in Mice

Mice are anesthetized with a combination of ketamine (100 mg/kg i.p.) and xylazine (20 mg/kg i.p.) and placed in a stereotaxic head holder. A 10 μl Hamilton syringe fixed to a glass pipette (10-50 μM tip diameter) cab be used for preliminary studies that inject fluorescent markers and for studies that inject NSCs (Specific Aim 1.3). Visual inspection will be used to verify placement of the glass pipette. Next, 0.5-2 μl of the diffusable marker Alexa Fluor 488 (Molecular Probes) is injected into the scala media and tympani for epifluorescence confirmation of injection sites. Neurons in the spiral ganglion are labeled with Vybrant DiI labeling solution (Molecular Probes). Animals are allowed to recover for four days and then sacrificed. The middle ears of the mice are dissected out to reveal the bony cochlea and the distribution of the fluorescent markers are determined under observation with a Leica MZ FLIII stereomicroscope equipped with epifluorescence.

Transplantation of NSCs

NSC (C17.2 cells) are transplanted into the damaged regions of the organ of Corti in living mice. These cells will respond to cues in the surviving microenvironment of the sensory epithelium and will differentiate into hair cells to replace those that were lost. Other investigators have shown that by simply injecting neural stem cells through the round window into the scala tympani leads to the integration of a few cells into the undamaged neonatal rat organ of Corti and differentiation of what appeares to be limited numbers of hair cells (Ito et al., 2001). Our approach will be to implant clusters of C17.2 cells in a variety of locations within the cochlea to assess the most effective implantation techniques.

Groups of between 1,000 and 800,000 C17.2 derived NSCs are suspended in a 0.5 μl to 2 μl drop of phosphate buffer solution and drawn into the tip of a glass micropipette. This stem cell line contains the lacZ transgene that expresses B-gal in every cell to enable the identification of the transplanted cells following implantation into the host cochlea. The NSCs can be transplanted into the scala media, scala tympani, and spiral ganglion in the optimal conditions. Animals are divided into four groups: 1) mice receiving gentamicin followed by stem cell transplants into the left ear, 2) mice receiving gentamicin followed by equivalent volume of stem cell vehicle into the left ear, 3) mice receiving saline instead of gentamicin followed by stem cell transplants into the left ear, and 4) mice receiving saline and vehicle instead of gentamicin and stem cell transplants. Animals are sacrificed at 2 week intervals up to 8 weeks after transplantation.

The NSCs should be able to integrate into the surviving sensory epithelium of the host cochlea. Alternative to C17.2 cells line, embryonic stem cells that have been experimentally manipulated to express cochlear progenitor cell markers can also be used as our stem cell source. Additionally, another source of stem cells are cell lines established from umbilical chord and/or adult neural tissue.

Functional Recovery

From a functional point of view, the auditory system is an ideal model in which to measure the efficacy of stem cell replacement therapy. In comparison, the central nervous system (CNS) exhibits a high degree of intrinsic redundancy. Consequently, more than one behavioral result may become evident when a neuronal population is experimentally compromised. Therefore, the functional effectiveness of stem cell transplantation into the CNS becomes confounded by uncontrolled variables. The beauty of the auditory system is that we can measure both the loss and recovery of function by well established methods and with little interference from external variables.

The auditory brainstem response (ABR) is a well documented procedure that is used both clinically and experimentally to assess neuronal responses to auditory stimuli (Wang et al., 2001; Stevens, 2001). Tone pips can be used to specify ABR thresholds in the high, mid, and low frequency ranges. Gentamicin-induced hair cell loss would be associated with an increase in ABR thresholds. If stem cell transplants into the cochlea result in hair cell replacement, and these hair cells form connections with the VIII^{th} nerve, the ABR thresholds would improve to near normal levels. Therefore, the ABR would become a useful measurement of hair cell replacement.

All mice received baseline ABR threshold measurements prior to the administration of gentamicin/ethacrynic acid. The protocol for ABR testing is as follows: mice are anesthetized, placed in a sound booth, and a receiver is inserted in the external meatus. ABR potentials is evoked with tone pips and recorded via needle electrodes inserted through the skin. Stimuli are 5-ms pips. Sound level will be raised in 5 dB steps from roughly 10 dB below threshold up to 80 dB SPL. ABR thresholds is defined as the lowest sound level at which the response peaks are clearly present. Mice will be divided into four groups as described above and are re-tested for bilateral ABR thresholds prior to sacrifice. The degree of NSCs integration is verified histologically.

It is expected that an appropriate gentamicin protocol will result in an increase in ABR thresholds, with a greater threshold shift exhibited in the high frequencies. Transplanted stem cells are expected to result in functioning hair cells with established neural connections, and thereby an improvement in ABR thresholds in the ear that received the transplants is expected compare to the ear that did not receive the stem cells (negative control). However, when considering the proficiency of the NSCs to migrate, the contralateral ABRs may be affected as well. If the NSCs fail to develop into hair cells that have established neural connections, we would not expect to see a difference in ABR thresholds from that observed in sham transplant animals (mice that received gentamicin followed by NSC vehicle).

DPOAE Responses on Deafened Mice

Distortion-product otoacoustic emissions (DPOAEs) are used both clinically (i.e. neonatal hearing screening) and experimentally as an assessment of outer hair cell function. When two pure tones are presented to the ear, a tone with a predictable frequency is emitted from the cochlea and may be recorded in the external auditory meatus. This otoacoustic emission is generated by oscillation of the outer hair cells in response to the pure tone stimuli. Although these emissions do not correlate well with hearing sensitivity, they are a good assessment of outer hair cell integrity. Stem cells transplanted into the deafened ear will form functioning outer hair cells and improve depressed DPOAE thresholds to near-normal levels.

Following the ABR measurements, the ear probe us changed to a probe developed in Dr. Liberman's laboratory that has been engineered to initiate and measure DPOAEs from mice (Yoshida et al., 2000; Yoshida & Liberman, 2000). Bilateral DPOAEs is measured using an acoustic system consisting of two sound sources and one microphone. Stimuli consist of two primary tones, a Fast Fourier Transform (FFT) of the response is computed, and the sound pressures at f₁, f₂ and 2f₁−f₂ is extracted after spectral averaging from serial waveform traces.

Deaf mice (or those that are induced to be deaf by gentamicin/EA treatment) do not have DPOAE thresholds. When the transplanted hair cells develop into outer hair cells, recovery of DPOAEs thresholds in the ear that received the stem cells is expected. A recovery of DPOAEs will provide evidence of functioning outer hair cells, and not estimate hearing thresholds or assess the establishment of synaptogenesis within the cochlea. Failure to find improvement in DPOAE responses will indicate that stem cells failed to integrate into the cochlea to form outer hair cells.

Effects of Stem Cell Transplantation on Electro-Mechanical Activity

It is possible that transplanted stem cells will not fully integrate into the cochlea, or that the NSCs will form only a few hair cells that establish functional connections. In this event, it is unlikely that any changes in ABR or DPOAE thresholds will be detected. Therefore, a more sensitive measurement of electrical activity would be required to measure any changes due to the integration of NSCs into the cochlea. To this end, round window recordings can be conducted to measure electrical changes caused by transplanted NSCs. The electrical activity recorded from the round window will be measured using established techniques (see General Methods; Yoshida et al., 2000; Yoshida & Liberman, 2000).

If transplanted NSCs successfully integrate into the cochlea, we expect the electrical activity at the round window in the stem cell treated ears to be similar to that recorded from control littermates. Because measurements at the round window are more sensitive than ABR or DPOAE measurements, it is possible to record changes in electrical activity at the round window without ABR or DPOAE responses. The absence of a change in electrical activity at the round window would indicate that the transplanted stem cells failed to form into functioning cochlear cells.

Morphology of New Hair Cells Derived From Transplanted Stem Cells in Regions of Hair Cell Loss in the Living Mammalian Cochlea

Regardless of the functional outcomes, a morphological assessment of stem cell integration into the organ of Corti is essential to document the fate of NSCs transplanted into the cochlea. Scanning electron microscopy and whole mount fluorescence/confocal microscopic imaging of the cochlea can be used to provide a comprehensive morphological assessment of stem cell fate.

Recent studies in the cortex have demonstrated that stem cells will only differentiate into neurons in areas where the native neurons have undergone apoptosis (Snyder et al., 1997). Work by Forge and colleagues (Li et al., 1995; Forge & Li, 2000; Forge & Schacht, 2000) and from our lab (Torchinsky et al., 1999) has shown that gentamicin kills hair cells by inducing apoptosis. Thus, elimination of hair cells from the mouse cochlea by gentamicin treatment should result in the appropriate microenvironment for inducing transplanted stem cells to differentiate as hair cells. In order to understand the potential for hair cell replacement by stem cells, it will be critical to first track the progression and extent of hair cell loss in the basal half of the mouse organ of Corti following gentamicin treatment.

To this end, host mice are administered an effective dose of gentamicin/EA to kill the hair cells. The mice are sacrificed at one and two weeks following the start of the treatment, and every two weeks after that through 8 weeks. The cochleae is dissected out and processed for scanning electron microscopy or whole mount fluorescence/confocal microscopy. The tissues are labeled with phalloidin and myosin VI or VIIa to identify the presence or absence of hair cells and the cytoarchitecture of the remaining supporting cells in the organ of Corti. The pattern and extent of gentamicin-induced hair cell loss along the length of the cochlear duct is assessed over the series of time points so that we know the onset of hair cell loss, how far it extends up the cochlea from the basal tip, when this damage reaches a maximum, and what the damaged epithelium looks like 8 weeks after the gentamicin treatment. In addition, the effects of hair cell loss on the innervation patterns in the organ of Corti is examined by labeling whole mount preparations with antibodies to either neurofilament proteins to label nerve processes (Ofsie & Cotanche, 1996) or to synapsin (Hennig & Cotanche, 1998) or neuron specific enolase (Whitlon & Sobkowicz, 1988) to label efferent and afferent synaptic terminals, respectively. These studies provide the baseline morphological controls for comparison to the animals which have had stem cell transplantations after gentamicin treatment.

We expect that the gentamicin injections will induce hair cell loss initially at the base of the cochlea and that this loss will spread more apically with time (Kotecha & Richardson, 1994; Forge & Schacht, 2000). We expect to see outer hair cell losses within the first week and total hair cell loss in the most basal regions of the cochlea by 2-4 weeks. As yet, it is unclear from previous studies how far up the cochlea the hair cell loss will extend with this dosage regimen in vivo, although studies in organ culture have demonstrated hair cell loss throughout three quarters of the entire organ of Corti at doses of 0.5-1 mM (Kotecha & Richardson, 1994). If gentamicin administration alone does not cause significant levels of hair cell loss throughout the cochlea in vivo, administering a combination of gentamicin and ethacrynic acid, which has been shown to increase the extent of hair cell loss in the cochlea without correlated nephrotoxicity, should achieve significant hair loss (Hayashida et al., 1989; Heil et al., 1992).

We expect that transplanted stem cells will migrate into the gentamicin-damaged region of the mouse cochlea and receive the required signals from the microenvironment, differentiate into new hair cells to replace those that were lost, and establish synaptic connections with the surviving cochlear nerve processes of the host cochlea. If so, then we should be able to identify these newly differentiating hair cells and follow their growth and establishment of neural connections with morphological analyses of the mouse cochlea using scanning electron microscopy and fluorescence/confocal microscopy.

Mice receiving stem cell transplants are distributed into the four groups described earlier and are sacrificed after the final physiological assessment. The cochleae are harvested and prepared for scanning electron microscopy or whole mount fluorescence/confocal microscopy examination. The basic surface morphology of the organ of Corti is determined with scanning electron microscopy and the cytoarchitecture of the sensory epithelium is assessed by labeling with phalloidin, a specific marker for filamentous actin. In addition, the presence or absence of hair cells in the lesioned region of the cochlea is examined with antibodies to either myosin VI or myosin VIIa, which are specific hair cell markers.

Evidence of newly differentiating hair cells is expected 2-4 weeks after stem cell transplantation in the cochleae of gentamicin-treated mice. These new hair cells should continue to differentiate and establish neural connections 4-8 weeks after the transplants. We would not expect to see any new hair cells in the cochleae of gentamicin-treated mice receiving transplants of the stem cell vehicle only. In addition, we would expect to see little, if any, new hair cells in the cochleae of mice not receiving gentamicin or in the cochleae of mice receiving saline and vehicle instead of gentamicin and stem cells. Ideally, the timing and pattern of new hair cell development in the gentamicin-treated mice stem cell transplants will be reflected by a parallel recovery of auditory function. However, it is possible that we would observe morphological hair cell recovery without a correlated functional recovery or one that precedes functional recovery by one or more weeks.

Stem cell transplants can be pursued using other sources of stem cells. For example, embryonic stem cells are thought to be totipotent and more amenable to integrating into whatever tissue they are placed in. We may use stem cells isolated from the umbilical cords of neonatal animals, or stem cells derived from adult hippocampus. Finally, we may also use cochlear stem cells, which are harder to obtain, but more capable of differentiating into cochlear structures.

Results

There is a growing body of evidence that points to the potential therapeutic applications of stem cells in diseases of the central nervous system, heart disease, and diabetes. The foundation of this work lies in the observation that stem cells are pluripotent and are able to differentiate into a variety of cell types depending on local environmental cues. We set out to determine if stem cells obtained from the brain had the ability to differentiate into cochlear cell types. To test this, we injected C17.2 neural stem cells (which stably express [beta]-galactosidase and GFP) into the round window of adult anesthetized FVB mice. We found that the C17.2 neural stem cells survived within the cochlear capsule for the duration of our experiments (4 weeks) and had migrated extensively throughout the cochlear duet. After 1 week, the spiral ganglion contained the highest concentration of stem cells suggesting a preference of the C17.2 neural stem cells for neural tissue. Immunohistochemistry revealed the upregulation of several spiral ganglion structural proteins including NF200, MAP-2, and [beta]-III tubulin, and co-localization with NT-3. Some of the transplanted C17.2 stem cells upregulated myosin 7a and parvalbumin as well. Next we asked whether the C17.2 neural stem cells would adopt an expression profile that more closely resembles that of the organ of Corri. To test this, we so-cultured C17.2 neural stem cells with OC-1 and OC-2 cochlear cell lines and used immunohistochemistry to identify the presence of cochlear specific proteins within the stem cells. We found that myosin 7a connexin 26, and parvalbumin were upregulated in the C17.2 neural stem cells after 7 days in co-culture with the cochlear cell lines. Furthermore, many of the C17.2 neural stem cells adopted the distinctive OC-1 and OC-2 phenotypes. These results indicate that neural stem cells possess the ability to transdifferentiate into cochlear cell types both in vitro and in vivo.

Results from experiments in which neural stem cells were transplanted into a deafened cochlea showed that the transplanted stem cells would migrate throughout the cochlea and adopt several phenotypic characteristics. In order to determine if transplanted neural stem cells had transdifferentiated into a cochlear cell or simply maintained a CNS profile, we set out to identify molecular markers that are selectively expressed in the cochlea and not expressed in neural stem cells. We compared the mRNA expression profiles of 96 genes of interest obtained from gene chip analysis of the whole mouse cochlea (Chen & Corey, JARO 3:140, 2002) to that obtained from C17.2 neural stem cells. We found 26 genes that were specific to the neural stem cells. We then surveyed the expression of these genes by immunohitochemistry and found 12 gene products (including NF200, connexin 26, MAP-2, parvalbumin, GluR2,3, and β-III tubulin) that are specific to the adult cochlea and one (Math1) that is specific to the C17.2 neural stem cells. These results elucidate adequate molecular markers that may be used to confirm the in vivo transdifferentiation of neural derived stem cells into cochlear cell types.

### REFERENCES

The references cited below and incorporated throughout the application are incorporated herein by reference.

1. Aboody, K., Brown, A., Rainov, N., Bower, K., Liu, S., Yang, W., Small, J., Herrlinger, U., Ourednik, V., PMcL, B., Breakefield, X. and E Y, S. 2000. Neural stem cells display extensive tropism for pathology in adult brain: Evidence from intracranial gliomas. PNAS 97, 12846-12851.

2. Avraham K B, Hasson T, Steel K P, Kingsley D M, Russell L B, Mooseker M S, Copeland N G, Jenkins N A: The mouse Snell's waltzer deafness gene encodes an unconventional myosin required for structural integrity of inner ear hair cells. Nature Genetics 1995;11:369-375

3. Brustle, O., Jones, K. N., Learish, R. D., Karram, K., Choudhary, K., Wiestler, O. D. Duncan, I. D. and McKay, R. D. G. (1999) Embryonic stem cell-derived glial precursors: a source of myelinating transplants. Science 285:754-756.

4. Cotanche, D. A. 1999. Structural recovery from sound damage and aminoglycoside treatment in the avian cochlea: a review. Audiol. and Neuro-Otol. 1999;4:271-285.

5. Evans, M. J. and Kaufman, M. H. (1981). Establishment in culture of pluripotential cells from mouse embryos. Nature 292:154-156.

6. Forge A, Schacht J: Aminoglycoside antibiotics. Audiol Neurootol 2000;5:3-22.

7. Forge, A. and Li, L. 2000. Apoptotic death of hair cells in mammalian vestibular sensory epithelia. Hear Res 139, 97-115.

8. Hasson, T, Gillespie, P. G., Garcia, J. A., MacDonald, R. B., Zhao, Y., Yee, A. G., Mooseker, M. S. and Corey, D. P. (1997). Unconventional myosins in inner-ear sensory epithelia. J Cell Biol. 137:1287-1307.

9. Hayashida, T., Hiel, H., Dulon, D., Erre, J.-P., Guilhaume, A. and Aran, J.-M. 1989. Dynamic changes following combined treatment with gentamicin and EA with and without acoustic stimulation. Acta Otolaryngol (Stockh) 108, 404-413.

10. Hennig, A. K. and Cotanche, D. A. 1998. Regeneration of cochlear efferent nerve terminals after gentamycin damage. J. Neurosci. 18, 3282-3296.

11. Hiel H, Schamel A, Erre J P, Hayashida T, Dulon D, Aran J M. (1992) Cellular and subcellular localization of tritiated gentamicin in the guinea pig cochlea following combined treatment with ethacrynic acid. Hear Res Jan;57(2): 157-65

12. Hudspeth A J: How hearing happens. Neuron 1997;19:947-950

13. Ito J, Kawaguchi S, Kojima K: Survival of neural stem cells in the cochlea. Acta Otolaryngol 2001; 121:140-142.

14. Kelley, M. W., Talreja, D. R., and Corwin, J. T. (1995). Replacement of hair cells after laser microbeam irradiation in cultured organs of corti from embryonic and neonatal mice. J Neurosci. 15, 3013-3026.

15. Kelley, M. W., Xu, X. M., Wagner, M. A., Warchol, M. E., and Corwin, J. T. (1993). The developing organ of Corti contains retinoic acid and forms supernumerary hair cells in response to exogenous retinoic acid in culture. Development 119, 1041-1053.

16. Kotecha, B. and Richardson, G. P. 1994. Ototoxicity in vitro: effects of neomycin, gentamicin, dihydrostreptomycin, amikacin, spectinomycin, neamine, spermine and poly-L-lysine. Hear Res 73, 173-184.

17. Li, L., Nevill, G. and Forge, A. (1995). Two modes of hair cell loss from the vestibular sensory epithelia of the guinea pig. J. Comp. Neurol. 355, 405-417.

18. Learish, R. D., Brustle, O., Zhang, S. C., and Duncan, I. D. (1999) Intraventricular transplantation of oligodendrocyte progenitors into a fetal myelin mutant results in widespread formation of myelin. Ann. Neurol. 46:716-722.

19. McDonald, J. W., Liu, X. Z., Qu, Y., Liu, S., Mickey, S. K., Turetsky, D., Gottlieb, D. I., and Choi, D. W. (1999) Transplanted embryonic stem cells survive, differentiate and promote recovery in injured rat spinal cord. Nat. Med. 5:1410-1412.

20. Martin, G. R. (1981). Isolation of a pluripotent cell line from early mouse embryos cultured in medium conditioned by teratocarcinoma stem cells. Proc. Natl. Acad. Sci. USA. 78:7634-7636.

21. Ofsie M S, Cotanche D A: Distribution of nerve fibers in the basilar papilla of normal and sound-damaged

22. chick cochleae. J. Comp. Neurol. 1996;370:281-294

23. Ourednik V, Ourednik J, Flax J D, Zawada M, Hutt C, Yang C, Park K I, Kim S U, Sidman R L, Freed C R,

24. Snyder E Y. (2001) Segregation of Human Neural Stem Cells in the Developing Primate Forebrain. Science. Jul 26 [epub ahead of print]

25. Rehm, H. L. and C. C. Morton (1999). “A new age in the genetics of deafness.” Genetics in Medicine 1: 295-302.

26. Snyder, E., Deitcher, D., Walsh, C., Arnold-Aldea, S., Hartwig, E. and Cepko, C. 1992. Multipotent neural cell lines can engraft and participate in development of mouse cerebellum. Cell 68, 33-51.

27. Snyder, E., Taylor, R. and Wolfe, J. 1995. Neural progenitor cell engraftment corrects lysosomal storage throughout the MPS VII mouse brain. Nature 374, 367-370.

28. Snyder, E., Yoon, C., Flax, J. and J D, M. 1997. Multipotent neural precursors can differentiate toward replacement of neurons undergoing targeted apoptotic degeneration in adult mouse neocortex. PNAS 94, 11663-11668.

29. Stevens J. (2001) State of the art neonatal hearing screening with auditory brainstem response. Scand Audiol Suppl (52):10-2

30. Stone, J. and Rubel, E. 2000. Cellular studies of auditory hair cell regeneration in birds. PNAS 97, 11714-11721.

31. Torchinsky, C., Messana, E. P., Arsura, M. and Cotanche, D. A. 1999. Regulation of p27Kip1 during gentamicin mediated hair cell death. J. Neurocytol. 28, 913-924.

32. Wang C C, Tien H C, Hsu C Y. (2001). Diagnosis and treatment of lipomas of the internal auditory canal. Ear Nose Throat J (5):340-2, 345

33. Whitlon DS, Sobkowicz H M: Neuron-specific enolase during the development of the organ of Corti. Int. J. Dev. Neurosci. 1988;6:77-87.

34. Yandava, B., Billinghurst, L. and Snyder, E. 1999. “Global” cell replacement is feasible via neural stem cell transplantation: Evidence from dysmyelinated shiverer mouse brain. PNAS 96, 7029-7034.

35. Yoshida, N. and Liberman, M. 2000. Sound conditioning reduces noise-induced permanent threshold shift in mice. Hear Res 148, 213-219.

36. Yoshida, N., Hequembourg, S., Atencio, C., Rosowski, J. and Liberman, M. 2000. Acoustic injury in mice: 129/SvEv is exceptionally resistant to noise induced hearing loss. Hear Res 141, 97-106.

We claim:

1. A method of treating sensorineural hearing loss in a subject in need thereof comprising administering to said subject a therapeutically effective amount of stem cells, capable of differentiating into cochlear hair cells, to an inner ear of a subject in need of such treatment.
